(19) United States
(12) Patent Application Publication
(10) Pub. No.: US 2003/0004089 A1
(43) Pub. Date: Jan. 2, 2003

(54) Perfume tester comprising a test strip

(76) Inventors: Hans-Peter Huber, Ostringen (DE); Wolfram Schulze, Stutensee (DE); Herbert Schuhrer, Bruchsal (DE)

Correspondence Address: BROWDY AND NEIMARK, P.L.L.C., 624 NINTH STREET, NW, SUITE 300, WASHINGTON, DC, 20001-5303, US

(21) Appl. No.: 10/203,801

(22) PCT Filed: Nov. 16, 2001
(86) PCT No.: PCT/DE01/04316

(30) Foreign Application Priority Data
Nov. 25, 2000 (DE) 200 20 049.6

(51) Int. Cl. A61K 7/46; G01N 31/22

(52) U.S. Cl. 512/1; 422/56; 422/68.1; 422/69

(57) ABSTRACT

A perfume tester comprises a test strip made of an absorbent material that is wetted with perfume. An end area of the test strip (3) is fixed inside a pouch (1) made of plastic films (1.1, 1.2). Above this end area, the pouch (1) comprises a specified rupture line (6), which extends over the width of the pouch and which tears along the longitudinal axis of the test strip (3) when the ends (4.5) of the pouch (1) are subjected to tensile stress thereby exposing a significant portion of the test strip (3). The inventive perfume tester can be easily produced and is well-suited for all channels of distribution. The impregnated test strip is protected against direct finger contact thereby enabling several successive smelling tests to be carried out without the test strip becoming adulterated.

### FIELD OF THE INVENTION

The invention relates to a perfume tester with a test strip of absorbent material that is moistened with perfume.

### PRIOR ART

To demonstrate perfumes to the user in terms of their fragrance note, perfume from original packages is usually applied to striplike blanks of absorbent paper. Waving this strip then wafts the fragrance through the air and has an influence on the decision to purchase.

Since saturating the test strip is always done on site, a demonstration can be done only directly in the sales area, but not conventionally by distributing samples or placing inserts in magazines.

Systems that allow this are very complicated and expensive to produce and only inadequately meet the demands made of them. Perfume testers produced by deep drawing do not, unless they have expensive additional parts, meet the demands that made of press samples.

### SUMMARY OF THE INVENTION

The invention has the object of realizing the time-tested principle of the saturated strip-type perfume tester with the aid of modern packaging and packing technology and developing a perfume tester that is functional to handle, favorable to produce, and furthermore suitable for all distribution paths (including inserts in periodicals) and offers enough surface area for printed advertising.

This object is attained according to the invention in that an end region of the test strip is fixed in a pouch of plastic films, and that above this end region, the pouch has a rated breaking line, extending over its width, which upon tensile stress of the ends of the pouch tears along the longitudinal axis of the test strip and uncovers a substantial portion of the test strip.

The design of the perfume tester, given appropriate handling, precludes direct contact of the fingers with the saturated test strip. This makes it possible for sniff tests of several different types of perfume to be performed in succession, without altering the fragrance notes.

### BRIEF DESCRIPTION OF THE DRAWINGS

Several exemplary embodiments of the perfume tester of the invention will now be described in further detail in conjunction with the drawings. Shown are:

FIG. 1: an elevation view of the first exemplary embodiment of the perfume tester;

FIG. 2: a longitudinal section through the perfume tester of FIG. 1;

FIGS. 3A and 3B: detail views of the rated breaking line;

FIG. 4: an illustration of the handling; and

FIG. 5: two views of a second exemplary embodiment;

FIGS. 6, 6A: views of a third exemplary embodiment;

FIGS. 7, 7A: views of a fourth exemplary embodiment;

FIG. 8: views of a fifth exemplary embodiment;

FIG. 9: views of a sixth exemplary embodiment.

### DESCRIPTION OF THE EXEMPLARY EMBODIMENTS

From a film that is resistant to the perfume used (such as PET/Barex or PET/Alu/Barex), a pouch 1 is shaped out of two films 1.1 and 1.2 and is provided with a tight weld 2 all the way around by means of sealing seams 2.1 . . . 2.4. A test strip 3 is introduced around the resultant free space in such a way that its first end region is located closer to the sealing seam 2.1 than the second end region is to the sealing seam 2.3.

The test strip 3 can already be saturated with perfume before being introduced into the pouch 1; it is also possible to introduce the perfume once there are three closed sealing seams 2.1, 2.2 and 2.4, to perfume the final sealing after that by means of the sealing seam 2.3.

The films 1.1 and 1.2 that form the pouch 1 serve to receive an informative printed advertising, which can include further instructions on handling.

A semicircular marking is located on the upper end 5; it is applied by printing, for instance, and is intended to visually indicate a region that is to be held firmly.

In the region A/B (FIG. 3), a rated breaking line 6 is made on the outsides of each of the films 1.1 and 1.2, congruently in terms of position, and this line can be designed as a straight line or, as shown here, as an undulating line, depending on the film material used, which is also product-dependent.

In general, this rated breaking line 6 is embodied such that the outer layer 1.4 of the film composite 1.1/1.2 is severed (variant A). Depending on the film material used, it may become necessary for the layer 1.5 located under the outer layer 1.4 to be minimally scored at the same time (variant B).

The rated breaking line is made by laser cutting or mechanically with cutter blades.

At the end points of the rated breaking line 6, punched markings 7 can be provided, to reinforce the visual indication of the location of the rated breaking line.

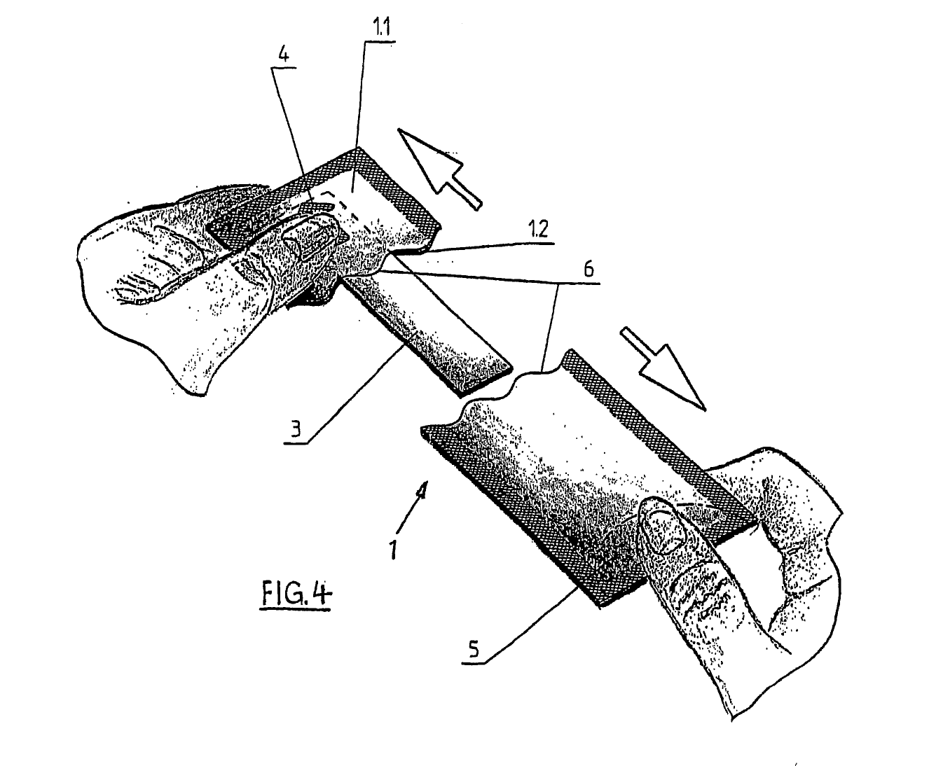

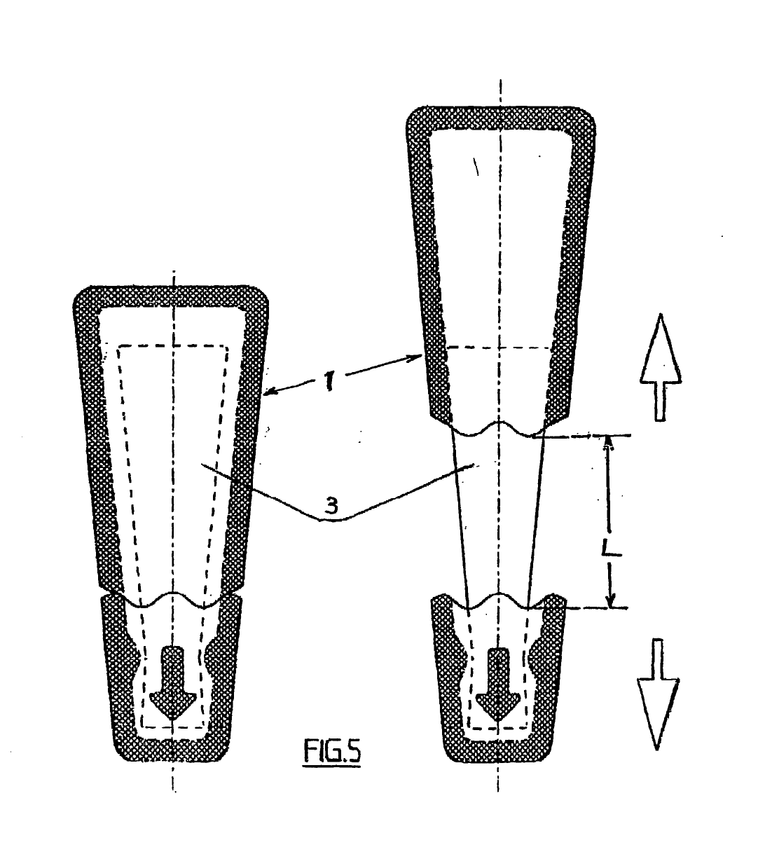

The handling of the perfume tester embodied in this way is shown in FIG. 4:

The pouch 1 is grasped with the fingers at the marked ends 4 and 5 and subjected to a tensile stress. As a result, the films 1.1 and 1.2 tear at the rated breaking line 6, and the perfume-saturated test strip 3 is uncovered; the sniff test can begin.

After use, the free end of the test strip 3 can be thrust back into the detached part of the pouch 1.

One part of the pouch 1 (the one having the end 5) can be pulled completely off the test strip, as shown in FIG. 4, or only partly, which makes it easier to push the pouch together after the sniff test and in particular reliably prevents someone from grasping the test strip.

For the latter purpose, in a second exemplary embodiment, the outer contours of the test strip 3 and the inner contours of the sealing seams can for instance be embodied conically in pairs in such a way that a stop is formed once a certain pulled-out length L of the pouch 1 is reached (FIG. 5).

The way in which the test strip 3 is fixed in the locking end 4 of the pouch 1 will now be described in terms of several exemplary embodiments. Otherwise, the handling of the perfume tester is the same for all the exemplary embodiments.

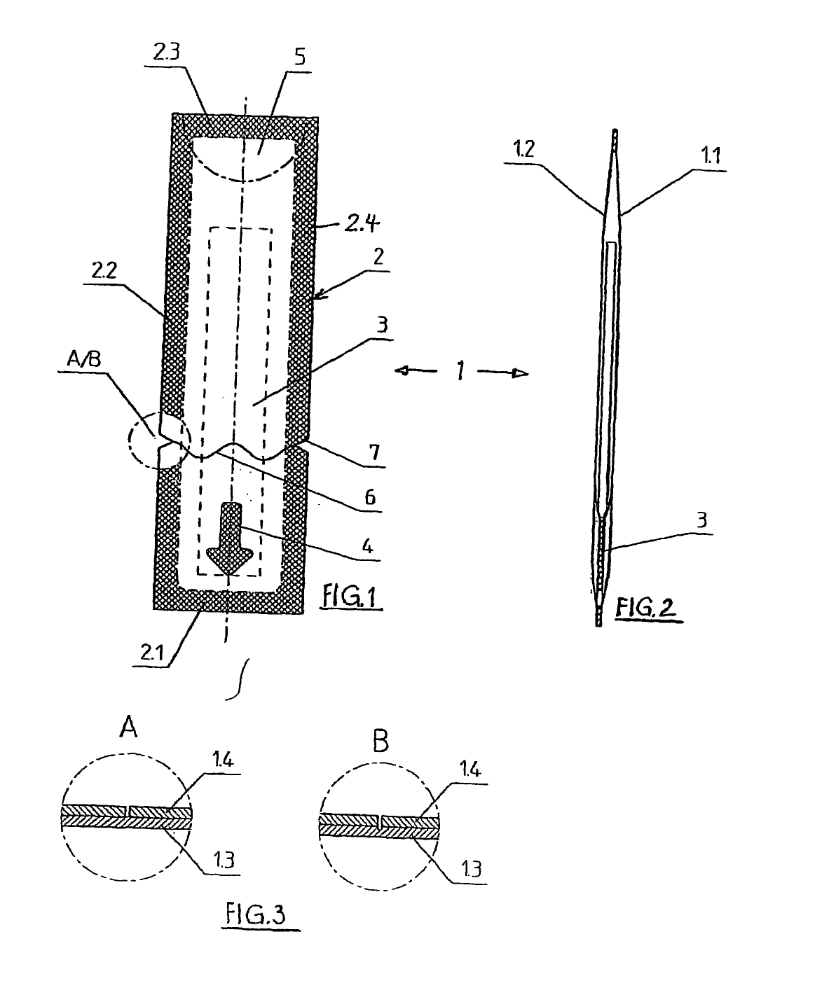

In the first and second exemplary embodiments (FIGS. 1-5), the locking end is fixed in the pouch 1 by means of a heated sealing die in the first end region of the test strip 3. By it shape, the sealing die used can also—as indicated

in the form of an arrow—provide some indication pertaining to opening. However, this kind of locking depends greatly on the test strip materials used.

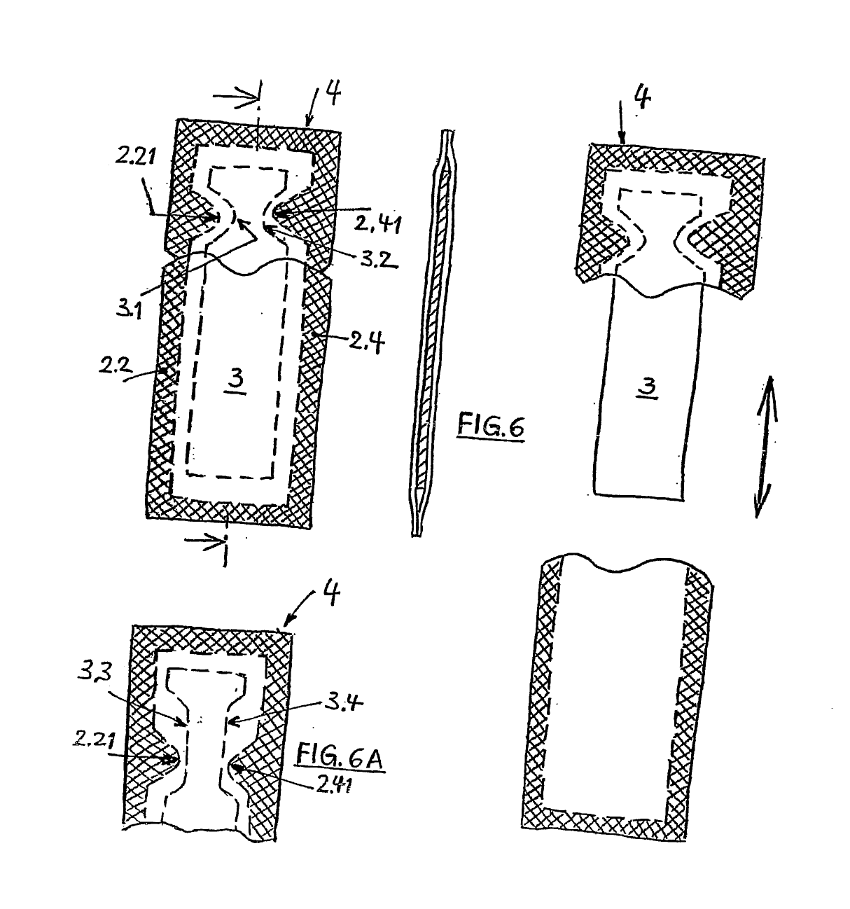

The other exemplary embodiments (FIGS. 6-9) therefore show possible ways of enabling the fixation of the test strip 3 in the locking end 4 of the pouch 1 not by sealing but rather by positive engagement between the sealing regions and the test strip 3:

In the third exemplary embodiment (FIGS. 6, 6A), the test strip 3 has lateral indentations 3.1, 3.2, into which inward-oriented sealing protrusions 2.21, 2.41 of the longitudinal seals 2.2, 2.4 extend, so that when the test strip 3 is pulled out, it is kept practically stationary in the locking end 4. If a certain play of motion is to be allowed to the test strip 3, then longer indentations 3.3, 3.4 (FIG. 6A) can be provided, so that the narrowed portion of the test strip 3 can be displaced along the sealing protrusions 2.21, 2.41.

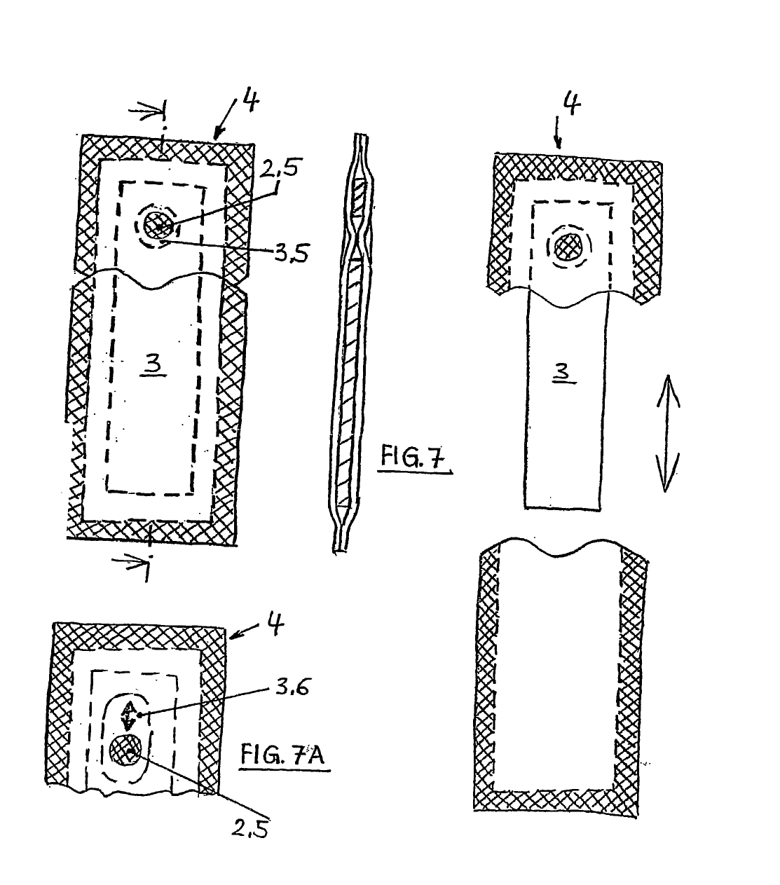

In the fourth exemplary embodiment (FIGS. 7, 7A), the test strip 3 has a recess 3.5 in the locking end 4, through which recess a sealing face 2.5 is “sealed through”, so that once again the desired locking effect is obtained as a result. If a limited displacement is to be allowed here, then embodying the recess as an oblong slot 3.6 (FIG. 7A) is suggests itself.

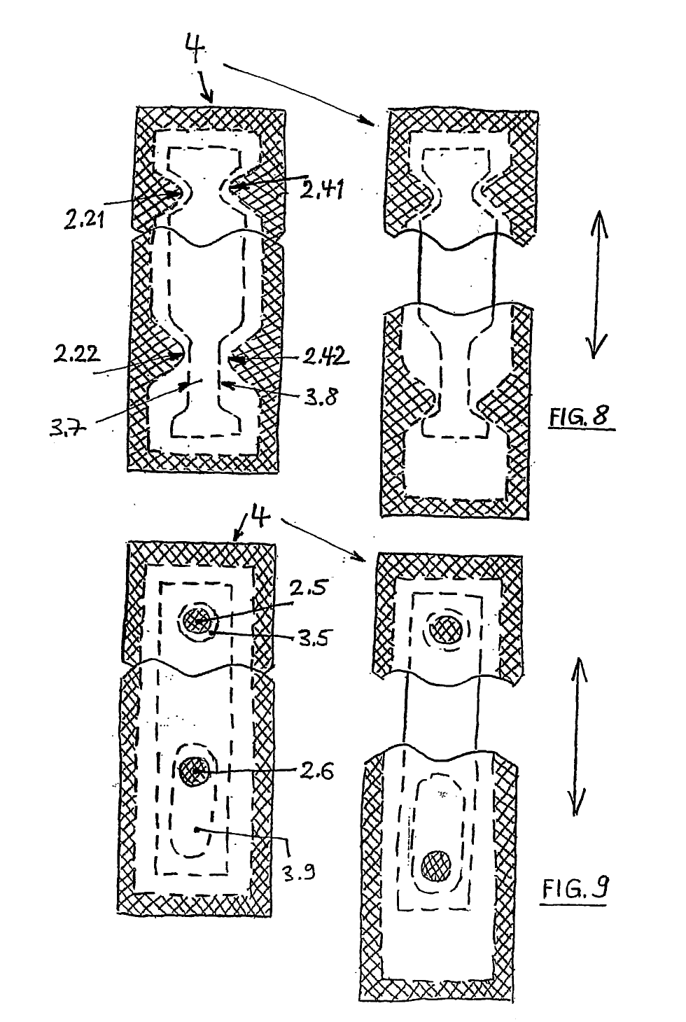

In the fifth exemplary embodiment (FIG. 8), the locking of FIG. 6 is linked with a limitation of the pulling-out distance by means of further sealing protrusions 2.22, 2.42 located opposite indentations 3.7, 3.8; in the sixth exemplary embodiment (FIG. 9), the locking of FIG. 7 is linked with a limitation of the pull-out distance by means of a further oblong-slot recess 3.9, through which a sealing face 2.6 extends.

1. A perfume tester having a test strip of absorbent material that is moistened with perfume,
characterized in that an end region of the test strip (3) is fixed in a pouch (1) of plastic films (1.1, 1.2), and that above this end region, the pouch (1) has a rated breaking line (6), extending over its width, which upon tensile stress of the ends (4, 5) of the pouch (1) tears along the longitudinal axis of the test strip (3) and uncovers a substantial portion of the test strip (3).

2. The perfume tester of claim 1, characterized in that the rated breaking line (6) is made in at least one of the two plastic films (1.1, 1.2).

3. The perfume tester of claim 2, characterized in that at least one of the lateral end points of the rated breaking line (6) in the peripheral region (2) is embodied as a punched marking (7).

4. The perfume tester of claim 1, characterized in that the fixation of the test strip (3) in the pouch (1) is effected by sealing.

5. The perfume tester of claim 1, characterized in that the fixation of the test strip (3) in the pouch (1) is effected by positive engagement of corresponding contours of the test strip (3) and pouch (1).

6. The perfume tester of claim 5, characterized in that the contours are formed of lateral indentations (3.1, 3.2; 3.3, 3.4) of the test strip (3) and opposed sealing protrusions (2.21, 2.41) of the sealing edge (2) of the pouch (1).

7. The perfume tester of claim 5, characterized in that the contours comprise at least one recess (3.5) in the test strip (3), through which a sealing region (2.4) of the pouch (1) extends.

8. The perfume tester of claim 6 or 7, characterized in that the shape of the indentations (3.1, 3.2; 3.3, 3.4) or of the recess (3.5) permits a limited longitudinal displacement of the test strip (3) in the locking end (4).

9. The perfume tester of claim 1, characterized in that the internal contour of the sealing seams (2.1, 2.2, 2.3, 2.4) connecting the two films (1.1, 1.2) and the outer contour of the test strip (3) are adapted to one another such that after the rated breaking line (6) breaks, the unfixed part of the pouch is completely detachable from the test strip (3).

10. The perfume tester of claim 1, characterized in that the internal contour of the sealing seams (2.1, 2.2, 2.3, 2.4) connecting the two films (1.1, 1.2) and the outer contour of the test strip (3) are adapted to one another such that after the rated breaking line (6), the two parts of the pouch (1) uncover only a middle portion of the test strip (3).
